SEQUENCE LISTING (9 sequences)

SEQ ID NO: 1 - protein, 21 aa, Artificial Sequence
  Description of Artificial Sequence Synthetic peptide
TFGGAXGFPLGSPLXSPVFPR

SEQ ID NO: 2 - protein, 21 aa, Canis lupus
TFGGAGGFPLGSPLGSPVFPR

SEQ ID NO: 3 - protein, 21 aa, Canis lupus
  MOD_RES (12)..(12): Phospho-Ser
TFGGAGGFPLGSPLGSPVFPR

SEQ ID NO: 4 - protein, 21 aa, Unknown
  Description of Unknown Desmin peptide
TFGGAGGFPLGSPLGSPVFPR

SEQ ID NO: 5 - protein, 83 aa, Homo sapiens
MSQAYSSSQRVSSVRRTFGGAPGFPLGSPLSSPVFPRAGFGSKGSSSSVTSRVYQVSRTSGGAGGLGSLRASRLGTTRTPSSY

SEQ ID NO: 6 - protein, 82 aa, Canis lupus
MSQAYSSSQRVSSVRRTFGGAGGFPLGSPLGSPVFPRAGFGTKGSSSSVTSRVYQVSRTSGGAGGLGALRAGRLGTGRAPSY

SEQ ID NO: 7 - protein, 21 aa, Canis lupus
  MOD_RES (12)..(12): Phospho-Ser
TFGGAGGFPLGSPLGSPVFPR

SEQ ID NO: 8 - protein, 23 aa, Homo sapiens
RTFGGAPGFPLGSPLSSPVFPRA

SEQ ID NO: 9 - protein, 23 aa, Homo sapiens
  MOD_RES (13)..(13): Phospho-Ser
RTFGGAPGFPLGSPLSSPVFPRA

(19) United States
(12) Patent Application Publication
(10) Pub. No.: US 2012/0303083 A1
(43) Pub. Date: Nov. 29, 2012

(54) NOVEL DESMIN PHOSPHORYLATION SITES USEFUL IN DIAGNOSIS AND INTERVENTION OF CARDIAC DISEASE

(75) Inventors: Giulio Agnetti, Baltimore, MD (US); Jennifer Van Eyk, Baltimore, MD (US)

(73) Assignee: The Johns Hopkins University, Baltimore, MD (US)

(21) Appl. No.: 13/322,760

(22) PCT Filed: May 26, 2010
(86) PCT No.: PCT/US2010/036228

§ 371 Date: Nov. 28, 2011

Related U.S. Application Data
(60) Provisional application No. 61/181,008, filed on May 26, 2009.
(60) Provisional application No. 61/265,970, filed on Dec. 2, 2009.

(51) Int. Cl. G01N 33/566; G01N 27/62; A61N 1/365; A61K 38/49; A61P 9/04; A61P 9/00; C07K 16/18; G01N 21/64

(52) U.S. Cl. 607/17; 530/389.1; 530/388.2; 530/391.3; 435/7.92; 435/7.1; 435/23; 424/94.64

(57) ABSTRACT

This invention relates to novel phosphorylation sites in the desmin protein that are associated with the onset of heart failure. The phosphorylation sites, i.e., Ser-27 and Ser-31, can be used as biomarkers for (i) identifying subjects at risk for the development of heart failure, (ii) treating subjects having a higher than normal level of the biomarker, and (iii) monitoring therapy of a subject at risk for the development of heart failure. Also described are antibodies, reagents, and kits for carrying out a method of the present invention.

This application claims the benefit of the filing date of provisional patent application Nos. 61/181,008, filed May 26, 2009, and 61/265,970, filed Dec. 2, 2009, which are incorporated by reference in their entirety herein.

The work leading to the invention described and claimed herein was carried out using funds from the National Institutes of Health and the National Heart, Lung, and Blood Institute, grant no, P01-HL077180. The U.S. Government has certain rights in the invention.

### FIELD OF INVENTION

The invention relates to novel phosphorylation sites in desmin, a protein associated with the development of heart failure.

### BACKGROUND INFORMATION

Heart failure (HF) is one of the most common causes of morbidity and mortality in Western societies, where it has a 5-years prognosis worse than any other malignancy. Diwan et al., Physiology 22:56-64 (2007). Despite the continuous efforts to find new effective therapies, the “pipeline” of drugs for HF is still running dry. Hoshijima et al., J Clin Invest 109:849-855 (2002); and Kass et al., Nat Med. 15:24-25 (2009). New technologies are needed to help refill that pipeline by providing new concepts and insights into the maladaptive mechanisms that regulate the transition to HF.

Desmin is a 52 kDa protein, and it is the protein component of intermediate filament cytoskeletons in myocytes. Capetanaki et al., Heart Fail Rev 5:203-220 (2000). Cardiac myocytes contain high levels of desmin, and several studies have shown that the levels of modified forms of desmin are changed in a number of cardiac conditions. Wang et al., Circ Res 99:1315-28 (2006). Previously, the quantitation of desmin in human heart failure was controversial, likely due to the existence of modified forms of the protein. Capetanaki et al., Heart Fail Rev 5:203-220 (2000); and Di Somma et al., Eur J Heart Fail 6; 389-98 (2004). We have identified the presence of posttranslationally modified (PTM) forms of desmin in viva. Specifically, we have discovered PTM-forms of desmin having decreased phosphorylation at Ser-27 and Ser-31 in subjects having heart failure.

Novel roles for desmin in the heart have been suggested in recent years. These include: differentiation of stem cells to cardiac myocytes (Holfrigi et al., Differentiation 75:616-26 (2007)); the regulation of organelle distribution and function, particularly mitochondrial (Capetanaki et al., Exp Cell Res 313:2063-76 (2007); and Milner et al., J Cell Biol 150:1283-98 (2000)); autophagy (Tannous et al., Proc Natl Acad Sci USA 105:9745-50 (2008)) and the formation of toxic amyloid species (Wang et al., J Card Fail 8:S287-92 (2002); Wang et al., Circ Res 93:998-1005 (2003); and Wang et al., Circ Res 89:84-91 (2001)). The fact that desmin assembly is affected by protein phosphorylation (Rappaport et al., FEBS Lett 231:421-25 (1988)) indicates that phosphorylation of desmin at Ser-27 and Ser-31 plays a role in the molecular mechanism of the formation of amyloid species toxic to the heart. Wang et al., Circ Res 99:1315-28 (2006).

### DESCRIPTION OF THE DRAWINGS

FIG. 1 shows images of desmin cytoskeleton reorganization in heart failure. Tissue samples from the canine model of heart failure (DHF) were prepared for fluorescent microscopy; probed with anti-desmin antibody, phalloidin (actin) and DAPI (nuclei); and assessed by confocal imaging. Staining with the anti-desmin antibody (green) shows the redistribution of IF cytoskeleton in DHF compared to sham operated control (SO), DAPI (in blue) was used to stain nuclei whereas actin (in red) was probed with phalloidin. Sarcomere disarrangement was also observed in DHF compared to SO. Interestingly, desmin distribution at the intercalated discs and Z-bands (striation) is recovered with CRT (n≧3).

FIG. 2 shows how levels of desmin PTM-forms (posttranslationally modified forms) are altered in heart failure. Tissue specimens from failing (DHF) and sham operated (SO) canine hearts were subjected to IN-sequence fractionation and analyzed with DIGE. FIG. 2A shows a representative image of a DIGE gel containing SO (green), DHF (red), and internal standard (blue) samples. Several PTM-forms of desmin were identified by mass spectrometry, and are indicated by arrows in FIG. 28 (reproduced in grayscale in FIG. 2C). Image analysis shows that three PTM-forms of desmin, which are compatible with a mono-phosphorylated form, a bi-phosphorylated form, and a fragment of desmin (labeled m, b and f in FIG. 2C, respectively), are increased in DHF (2-fold, p<0.05; FIGS. 2D-2F).

FIG. 3 shows how levels of dephosphorylated and fragment forms of Desmin are increased during heart failure. Tissue specimens from failing (DHF), sham operated (SO), and CRT treated canine hearts were subjected to IN-sequence fractionation and analyzed with DICE. The internal standard was treated with alkaline phosphatase (AP) prior to DICE analysis. FIG. 3A shows a magnified area of a representative DICE gel used in a three-way comparison between DHF (Cy5, red), SO (Cy3, green), and AP treated internal standard (Cy2, blue). FIG. 3B displays the same experiment comparing DHF and CRT samples. The estimated number of phosphate groups (PGs) per each spot is displayed for clarity. Desmin species are encircled in the magnified greyscale image provided in FIG. 3C. A representative image of a 1D western blot analysis for desmin is also shown (FIG. 3D), along with histograms that display the changes in band volume, normalized to total protein signal/lane.

FIG. 4 shows how levels of desmin PIM-forms are changed in human heart failure. Tissue samples from human subjects with heart failure (HF) were analyzed with DIGE. FIG. 4A is a representative DICE image showing the comparison between HF (Cy5, red) and control (C, Cy3, green) individuals. Several PTM-forms of desmin were also identified by mass spectrometry, and are indicated by arrows in FIG. 4B (reproduced in grayscale in FIG. 4C). FIG. 4D shows the amount of tissue utilized for the analysis (˜3 mg). Image analysis shows that a mono-phosphorylated form, a tri-phosphorylated form, and a fragment of desmin (labeled m, t and f in FIG. 4C, respectively) are all increased with HF (FIGS. 4E-4G).

FIG. 5 depicts desmin phosphorylation sites that are altered during heart failure. FIG. 5A shows the canine and human sequences of desmin. The TFGGAXGFPLGSPLXSPVFPR peptide and residues 27 and 31 are highlighted. FIG. 5B is a representative MS/MS spectra showing bi-phosphorylated Desmin (Ser-27 and -31) from human samples. FIG. 5C is a representative MS/MS spectrum for the TFGGAGGFPLGSPLGSPVFPR (m/z 1089.8) peptide from canine samples, FIG. 5C shows the y- and b-ions series and relative m/z values. Observed ions are indicated in the spectrum by their b or y number and the loss of water (—H₂O) or water and phosphate (—H₃PO₄, neutral loss). Observed masses are underlined in the list of m/z values of the ion-series as well. The MS/MS spectrum results indicate that desmin is phosphorylated at Ser-27 and Ser-31 in canines and humans in vivo.

FIG. 6 shows the results of a multiple reaction monitoring (MRM) experiment with human desmin. FIG. 6A is a schematic illustration of an MRM experiment. FIG. 6B is a representative MRM spectra of human clinical samples that were collected and assessed for the presence of un-phosphorylated (m/z=2087.91) and mono-phosphorylated (2166.69) desmin peptide.

FIG. 7 shows the identification of desmin-positive amyloid oligomers during heart failure. FIG. 7A is a representative image of a blue-native PAGE gel showing the desmin oligomers present in the myofilament enriched fraction. FIG. 7B is a representative western blot using an anti-desmin antibody. FIG. 7C shows the normalized values for the volumes of the desmin bands at 200 kDa. FIG. 7D is a magnified image of a representative western blotting using an anti-A11 oligomer antibody. FIG. 7E depicts the results of the densitometric analysis of the western blotting using the anti-A11 oligomer antibody.

### DESCRIPTION

The present invention is directed to novel phosphorylation sites in desmin, which is a protein component of intermediate filaments (IFs) in cardiac myocytes. The present inventors have demonstrated that certain forms of desmin are present in subjects having heart failure. Specifically, the present inventors have discovered that a modified form of desmin having decreased levels of phosphorylation at Ser-27 and Ser-31 is present during heart failure.

Accordingly, in some embodiment of the present invention, it is desirable to use desmin phosphorylation at Ser-27 and/or Ser-31 as a biomarker to identify a subject at risk for developing heart failure. In some embodiments, a sample is obtained from the subject and the biomarker is detected using a conventional detection method(s) that is well-known in the art. In some embodiments, the biomarker is identified by immunoassay or mass spectrometry. In embodiments, the biomarker is identified by ELISA or immunohistochemistry. In embodiments, the biomarker is detected by Multiple Reaction Monitoring (MRM). In some embodiments, the biomarker is detected by two-dimensional electrophoresis (2DE, separating proteins based on pI and molecular weight), two-dimensional liquid chromatography (2DLC, separating proteins based on pI and hydrophobicity), or one-dimensional liquid chromatography (1DLC, separating proteins based on hydrophobicity). In some embodiments, the biomarker is detected by electron microscopy.

Another aspect of the present invention is a method for deciding how to treat a subject suspected of having heart failure, or a subject that is at high risk for developing heart failure. In some embodiments, a sample is obtained from the subject and the biomarker is detected using conventional detection methods that are well-known in the art. The sample is then compared to a baseline/normal level of desmin phosphorylation. In some embodiments, a subject having decreased levels of desmin phosphorylation at Ser-27 and/or Ser-31 is determined to have (or is likely to have) heart failure, and is treated with aggressive therapy [such as cardiac resynchronization therapy; heart valve repair or replacement; implantable cardioverter-defibrillator; heart pump; heart transplant; percutaneous coronary intervention (i.e., angioplasty); coronary bypass surgery to replace the injured/blocked coronary artery; or administration of an angiotensin-converting enzyme (ACE) inhibitor, angiotensin receptor blocker (ARE), digoxin, beta blockers, diuretics, or aldosterone antagonist]. In some embodiments, a subject having normal levels of desmin phosphorylation at Ser-27 and/or Ser-31 is determined not to have (or is not likely to have) heart failure, and is treated with non-aggressive therapies [such as administration of asprin and thrombolysis (e.g., TPA), with periodic monitoring to ensure no future cardiac events; or by recommending changes in life style].

In one embodiment of the invention, the phosphorylation state of Ser-27 and/or Ser-31 in the desmin protein is compared over time to a baseline/normal value and/or to levels known to be associated with heart failure. The kinetic rise and fall of desmin phosphorylation is indicative of impending heart failure. In some embodiments, the level of desmin phosphorylation at Ser-27 and/or Ser-31 is compared over time in a subject receiving treatment. In some embodiments, the baseline value can be based on earlier measurements taken from the same subject, before the treatment was administered.

A method as described above may further comprise measuring in the sample the amount of one or more other markers that have been reported to be diagnostic of heart failure, including cardiac specific isoforms of troponin I (TnI) and/or troponin T (TnT), creatine kinase-MB (CK-MB), myoglobin, or brain natriuretic peptide (BNP). A significant increase (e.g., at least a statistically significant increase) of the one or more markers is further indicative that the subject is at risk for developing heart failure.

The present invention also provides antibodies that specifically bind to desmin at Ser-27. In some embodiments, the antibodies specifically bind to un-, mono-, bi-, and/or tri-phosphorylated Ser-27. In some embodiments, the antibodies are labeled. In some embodiments, the antibodies are labeled with a fluorescent moiety, a moiety that binds a reporter ion, a heavy ion, a gold particle, or a quantum dot.

The present invention provides antibodies that specifically bind to desmin at Ser-31. In some embodiments, the antibodies specifically bind to un-, mono-, bi-, and/or tri-phosphorylated Ser-31. In some embodiments, the antibodies are labeled. In sonic embodiments, the antibodies are labeled with a fluorescent moiety, a moiety that binds a reporter ion, a heavy ion, a gold particle, or a quantum dot.

The present invention also provides a method of detecting the phosphorylation state of desmin at Ser-27 and Ser-31 using conventional detection methods that are well-known in the art. In some embodiments, the method comprises using an antibody that specifically binds to phosphorylated desmin at Ser-27 and/or Ser-32. In some embodiments, the antibodies specifically bind to un-, mono-, bi-, and/or tri-phosphorylated Ser-27. In some embodiments, the antibodies specifically bind to un-, mono-, bi-, and/or tri-phosphorylated Ser-31. In some embodiments, the antibodies are labeled. In some embodiments, the antibodies are labeled with a fluorescent moiety, a moiety that binds a reporter ion, a heavy ion, a gold particle, or a quantum dot.

Another aspect of the invention is a kit for identifying a subject at risk for developing heart failure. In some embodiments, the kit contains an agent that detects the phosphorylation state of desmin at Ser-27 and/or Ser-31. In some embodiments, the kit contains an antibody that detects the level of desmin phosphorylation at Ser-27 and/or Ser-31. In some embodiments, the antibody specifically binds to un-, mono-, bi-, and/or tri-phosphorylated Ser-27. In some embodiments, the antibody specifically binds to un-, mono-, bi-, and/or tri-phosphorylated Ser-31. In some embodiments, the antibody is labeled. In some embodiments, the antibody is labeled with a fluorescent moiety, a moiety that binds a reporter ion, a heavy ion, a gold particle, or a quantum dot.

In some embodiments, the sample is analyzed by mass spectrometry. As such, in some embodiments, the kit contains labeled peptides (synthetic or recombinant).

To facilitate an understanding of the present invention, a number of terms and phrases are defined below.

As used herein, the singular forms “a”, “an”, and “the” include plural forms unless the context clearly dictates otherwise. Thus, for example, reference to “a protein” includes reference to more than one protein.

As used herein, “heart failure” refers to a condition in which a subject experiences inadequate blood flow to fulfill the needs of the tissues and organs of the body. Heart failure has been classified by the New York Heart Association (NYHA) into four classes of progressively worsening symptoms and diminished exercise capacity. Class I corresponds to no limitation wherein ordinary physical activity does not cause undue fatigue, shortness of breath, or palpitation. Class II corresponds to slight limitation of physical activity wherein such patients are comfortable at rest, but wherein ordinary physical activity results in fatigue, shortness of breath, palpitations or angina. Class III corresponds to a marked limitation of physical activity wherein, although patients are comfortable at rest, even less than ordinary activity will lead to symptoms. Class IV corresponds to inability to carry on any physical activity without discomfort, wherein symptoms of heart failure are present even at rest and where increased discomfort is experienced with any physical activity. As such, heart failure includes cardiac-related illnesses such as myocardial infarction, ischemic heart disease, hypertension, valvular heart disease, and cardiomyopathy.

A sample which is “provided” can be obtained by the person (or machine) conducting the assay, or it can have been obtained by another, and transferred to the person (or machine) carrying out the assay.

By a “sample” (e.g. a test sample) from a subject is meant a sample that might be expected to contain elevated levels of the protein markers of the invention in a subject having heart failure. Many suitable sample types will be evident to a skilled worker. In some embodiments, the sample is a blood sample, such as whole blood, plasma, or serum (plasma from which clotting factors have been removed). For example, peripheral, arterial or venous plasma or serum can be used. In some embodiments, the sample is urine, sweat, or another body fluid into which proteins are sometimes removed from the blood stream. In the case of urine, for example, the protein is likely to be broken down, so diagnostic fragments of the proteins of the invention can be screened for. In some embodiments, the sample is cardiac tissue, which is harvested, e.g., after a heart transplant or the insertion of a pacemaker or defibrillator. In some embodiments, the tissue is tissue slices or tissue homogenates. Methods for obtaining samples and preparing them for analysis (e.g., for detection of the amount of protein) are conventional and are well-known in the art.

A “subject,” as used herein, includes any animal that has, or is at risk of developing, heart failure. Suitable subjects (patients) include laboratory animals (such as mouse, rat, rabbit, guinea pig or pig), farm animals, sporting animals (e.g., dogs or horses), domestic animals, and pets (such as a horse, dog or cat). Non-human primates and human patients are included. For example, human subjects who present with chest pain or other symptoms of cardiac distress, including, e.g., shortness of breath, nausea, vomiting, sweating, weakness, fatigue, or palpitations, can be evaluated by a method of the invention. In addition, subjects not exhibiting these symptoms can also be evaluated by a method of the present invention. Some subjects at risk for developing heart failure (e.g., subjects with myocardial infarction) do not experience symptoms such as chest pain. Furthermore, patients who have been evaluated in an emergency room, in an ambulance, or in a physician's office and are dismissed as not being ill according to current tests for heart failure can have an increased risk of having a heart attack in the next 24-48 hours. Such patients can be monitored by a method of the invention to determine if and when they begin to express markers of the invention, indicating that the subject is now at risk for developing heart failure. Subjects can also be monitored by a method of the invention to improve the accuracy of current provocative tests for assessing the risk of developing heart failure, such as exercise stress testing. An individual can be monitored by a method of the invention during exercise stress tests to determine if the individual is at risk for developing heart failure; such monitoring can supplement or replace the test that is currently carried out. Athletes (e.g., humans, racing dogs or race horses) can be monitored during training to ascertain if they are exerting themselves too vigorously and are in danger of developing heart failure.

“At risk of” is intended to mean at increased risk of, compared to a normal subject, or compared to a control group, e.g., a patient population. Thus, a subject carrying a particular marker may have an increased risk for a specific disease or disorder, and be identified as needing further testing. “Increased risk” or “elevated risk” mean any statistically significant increase in the probability, e.g., that the subject has the disorder.

Although much of the data presented in the Examples herein are directed to particular forms of desmin (or peptides thereof), it will be evident to a skilled worker that a variety of forms of these proteins may be indicative of the risk of developing heart failure in a subject. For example, the protein may be an intact, full-length desmin. In addition, as discussed in detail below, degraded and/or fragmented forms of desmin are also associated with heart failure. In such a case, an investigator can determine the level of one or more of the fragments or degradation products. Furthermore, when desmin undergoes processing naturally (e.g., posttranslational modifications, such as acetylation, methylation, phosphorylation, etc.), any of these forms of the protein are included in the invention. As such, “desmin” refers to full-length desmin, a fragment of desmin, and posttranslationally modified forms of desmin.

A variety of tests have been used to detect heart failure. These include, e.g., determining the levels of cardiac specific isoform(s) of troponin I (TnI) and/or troponin T (TnT), CK-MB (Creatine Kinase-MB), myoglobin, and brain natriuretic peptide (BNP). However, none of these markers is completely satisfactory for the detection of heart failure. For example, they can fail to detect early stages of heart failure, such as non-necrotic myocardial ischemia. The new markers described herein can be used in conjunction with these types of assays.

When the values of more than one protein are being analyzed, a statistical method such as multi-variant analysis or principal component analysis (PCA) is used which takes into account the levels of the various proteins (e.g., using a linear regression score). For verification, we will use either an immunoassay or a multiple reaction monitoring (MRM, a MS-based targeted method that quantifies peptides that are unique to the protein of interest).

In some embodiments, it is desirable to express the results of an assay in terms of an increase (e.g., a statistically significant increase) in a value (or combination of values) compared to a baseline value.

A “significant” increase in a value, as used herein, can refer to a difference which is reproducible or statistically significant, as determined using statistical methods that are appropriate and well-known in the art, generally with a probability value of less than five percent chance of the change being due to random variation. In general, a statistically significant value is at least two standard deviations from the value in a “normal” healthy control subject. Suitable statistical tests will be evident to a person of ordinary skill in the art. For example, a significant increase in the amount of a protein compared to a baseline value can be about 50%, 2-fold, or more higher. A significantly elevated amount of a protein of the invention compared to a suitable baseline value, then, is indicative that a test subject has a risk of developing heart failure. A subject is “likely” to be at risk for developing heart failure if the subject has levels of the marker protein(s) significantly above those of a healthy control or his own baseline (taken at an earlier time point). The extent of the increased levels correlates to the % chance. For example, the subject can have greater than about a 50% chance, e.g., greater than about 70%, 80% 90%, 95% or higher chance, of developing heart failure. In general, the presence of an elevated amount of a marker of the invention is a strong indication that the subject has heart failure.

As used herein, a “baseline value” generally refers to the level (amount) of a protein in a comparable sample (e.g., from the same type of tissue as the tested tissue, such as blood or serum), from a “normal” healthy subject that does not have heart failure. If desired, a pool or population of the same tissues from normal subjects can be used, and the baseline value can be an average or mean of the measurements. Suitable baseline values can be determined by those of skill in the art without undue experimentation. Suitable baseline values may be available in a database compiled from the values and/or may be determined based on published data or on retrospective studies of patients' tissues, and other information as would be apparent to a person of ordinary skill implementing a method of the invention. Suitable baseline values may be selected using statistical tools that provide an appropriate confidence interval so that measured levels that fall outside the standard value can be accepted as being aberrant from a diagnostic perspective, and predictive of heart failure.

It is generally not practical in a clinical or research setting to use patient samples as sources for baseline controls. Therefore, one can use any of variety of reference values in which the same or a similar level of expression is found in a subject that does not have heart failure.

It will be appreciated by a person of ordinary skill in the art that a baseline or normal level need not be established for each assay as the assay is performed, but rather, baseline or normal levels can be established by referring to a form of stored information regarding a previously determined baseline levels for a given protein or panel of proteins, such as a baseline level established by using any of the methods described herein. Such a form of stored information can include, for example, a reference chart, listing or electronic file of population or individual data regarding “normal levels” (negative control) or positive controls; a medical chart for the patient recording data from previous evaluations; a receiver-operator characteristic (ROC) curve; or any other source of data regarding baseline levels that is useful for the patient to be diagnosed. In some embodiments the amount of the proteins in a combination of proteins, compared to a baseline value, is expressed as a linear regression score, as described, e.g., in Irwin, in Neter, Kutner, Nachtsteim, Wasserman (1996) Applied Linear Statistical Models, 4^{th} edition, page 295.

In some embodiments in which the progress of a treatment is being monitored, a baseline value can be based on earlier measurements taken from the same subject, before the treatment was administered.

The amount of a protein can be measured using any suitable method. Some methods involve the use of antibodies, binding ligands, or mass spectrometry tagged peptides specific for a protein of interest. Antibodies suitable for use in assays of the invention are commercially available, or can be prepared routinely. Methods for preparing and using antibodies in assays for proteins of interest are conventional, and are described, e.g., in Green et al., Production of Polyclonal Antisera, in Immunochemical Protocols, Manson ed. (Humana Press 1992); Coligan et al., in Current Protocols in Immunology, sections 2.4.1 and 2.5.1-2.6.7 (1992); Köhler & Milstein, Nature 256:495-7 (1975); and Harlow et al., Antibodies: A Laboratory Manual, page 726 (Cold Spring Harbor Laboratory Pub, 1988).

Immortalized human B lymphocytes immunized in vitro or isolated from an immunized individual that produce an antibody directed against a target antigen can be generated. See, e.g., Cole et al., Monoclonal Antibodies and Cancer Therapy, Alan R. Liss ed., p. 77 (1985); Boerner et al., J Immunol, 147 (1):86-95 (1991); and U.S. Pat. No. 5,750,373. Also, the human antibody can be selected from a phage library, where that phage library expresses human antibodies, as described, for example, in Vaughan et al., Na. Biotech, 14:309-314 (1996), Sheets et al., Proc Natl Acad Sci, 95:6157-6162 (1998), Hoogenboom and Winter, 1991, J. Mol. Biol., 227:381, and Marks et al., J Mol Biol, 222:581 (1991). Techniques for the generation and use of antibody phage libraries are also described in U.S. Pat. Nos. 5,969,108, 6,172,197, 5,885,793, 6,521,404; 6,544,731; 6,555,313; 6,582,915; 6,593,081; 6,300,064; 6,653,068; 6,706,484; and 7,264,963; and Rothe et al., J Mol Bio, J Mol Biol 376:1182-1200 (2007). Affinity maturation strategies, such as chain shuffling (Marks et al., Bio/Technology 10:779-783 (1992)), are known in the art and may be employed to generate high affinity human antibodies.

Humanized antibodies can also be made in transgenic mice containing human immunoglobulin loci that are capable upon immunization of producing the full repertoire of human antibodies in the absence of endogenous immunoglobulin production. This approach is described in U.S. Pat. Nos. 5,545,807; 5,545,806; 5,569,825; 5,625,126; 5,633,425; and 5,661,016.

Any of a variety of antibodies can be used in methods of the invention. Such antibodies include, e.g., polyclonal, monoclonal (mAbs), recombinant, humanized or partially humanized, single chain, Fab, and fragments thereof. The antibodies can be of any isotype, IgM, various IgG isotypes such as IgG₁, IgG₂ₐ, etc., and they can be from any animal species that produces antibodies, including goat, rabbit, mouse, chicken or the like. The term, an antibody “specific for” or that “specifically binds” a protein, means that the antibody recognizes a defined sequence of amino acids, or epitope in the protein. An antibody that is “specific for,” “specifically recognizes,” or that “specifically binds” a polypeptide refers to an antibody that binds selectively to the polypeptide and not generally to other polypeptides unintended for binding to the antibody. The parameters required to achieve such specificity can be determined routinely, using conventional methods in the art. Conditions that are effective for binding a protein to an antibody which is specific for it are conventional and well-known in the art.

“Detectable moiety” or a “label” refers to a composition detectable by spectroscopic, photochemical, biochemical, immunochemical, or chemical means. For example, useful labels include ³²P, ³⁵S, fluorescent dyes, electron-dense reagents, enzymes (e.g., as commonly used in an ELISA), biotin-streptavidin, dioxigenin, haptens and proteins for which antisera or monoclonal antibodies are available, or nucleic acid molecules with a sequence complementary to a target. The detectable moiety often generates a measurable signal, such as a radioactive, chromogenic, or fluorescent signal, that can be used to quantify the amount of bound detectable moiety in a sample. Quantitation of the signal is achieved by, e.g., scintillation counting, densitometry, flow cytometry, or direct analysis by mass spectrometry of intact or subsequently digested peptides (one or more peptide can be assessed). Persons of skill in the art are familiar with techniques for labelling compounds of interest, and means for detection.

In one embodiment of the invention, antibodies specific for a (one or more) protein of the invention are immobilized on a surface (e.g., are reactive elements on an array, such as a microarray, or are on another surface, such as used for surface plasmon resonance (SPR)-based technology, such as BIAcore), and proteins in the sample are detected by virtue of their ability to bind specifically to the antibodies. Alternatively, proteins in the sample can be immobilized on a surface, and detected by virtue of their ability to bind specifically to the antibodies. Methods of preparing the surfaces and performing the analyses, including conditions effective for specific binding, are conventional and well-known in the art.

Among the many types of suitable immunoassays are competitive and non-competitive assay systems using techniques such as BIAcore analysis, FACS analysis, immunofluorescence, immunohistochemical staining, Western blots (immunoblots), radioimmunoassays, ELISA, “sandwich” immunoassays, immunoprecipitation assays, precipitation reactions, gel diffusion precipitin reactions, immunodiffusion assays, agglutination assays, complement-fixation assays, immunoradiometric assays, fluorescent immunoassays, fluorescence-activated cell sorting (FACS), protein A immunoassays, etc. Assays used in a method of the invention can be based on colorimetric readouts, fluorescent readouts, mass spectrometry, visual inspection, etc. Assays can be carried out, e.g., with suspension beads, or with arrays, in which antibodies or cell or blood samples are attached to a surface such as a glass slide or a chip.

In one embodiment, a tissue sample (e.g. a cardiac tissue sample) is stained with a suitable antibody in a conventional immunohistochemical assay for those proteins which are present in the myocardium.

Mass spectrometry (MS) can also be used to determine the amount of a protein, using conventional methods. Some such typical methods are described in the Examples herein. Relative ratio between multiple samples can be determined using label free methods, based on spectral count (and the number of unique peptides and the number of observation of each peptide). Alternatively, quantitive data can be obtained using multiple reaction monitoring (MRM), most often carried out using a triple quadripole mass spectrometer. In this case, peptides that are unique to a given protein are selected in the MS instrument and quantified. Absolute quantification can be obtained if a known labeled synthetic peptide (e.g., ¹⁵N) is used. For detailed methods see, e.g., Qin Fu and J E Van Eyk, in Clinical Proteomics: from diagnostics to therapy, Van Eyk J E and Dunn M, eds, (Wiley and Son Press 2008); and Gundry et al., Preparation of Proteins and Peptides for Mass Spectrometry Analysis in a Bottom-Up Proteomics Workflow, Current Protocols in Molecular Biology, Ausubel et al. eds., (John Wiley & Sons, Inc., October 2009).

In general, molecular biology methods referred to herein are well-known in the art and are described, e.g., in Sambrook et al., Molecular Cloning: A Laboratory Manual, current edition, Cold Spring Harbor Laboratory, Cold Spring Harbor, N.Y., and Ausubel et al., Current Protocols in Molecular Biology, John Wiley & Sons, New York, N.Y.

“Diagnostic” means identifying the presence or nature of a pathologic condition and includes identifying patients who are at risk of developing a specific disease or disorder. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

A detection (diagnostic) method of the invention can be adapted for many uses. For example, it can be used to follow the progression of heart failure. In one embodiment of the invention, the detection is carried out both before (or at approximately the same time as), and after, the administration of a treatment, and the method is used to monitor the effectiveness of the treatment. A subject can be monitored in this way to determine the effectiveness for that subject of a particular drug regimen, or a drug or other treatment modality can be evaluated in a pre-clinical or clinical trial. If a treatment method is successful, the levels of the protein markers of the invention are expected to decrease.

As used herein, “treated” means that an effective amount of a drug or other anti-heart failure procedure is administered to the subject. An “effective” amount of an agent refers to an amount that elicits a detectable response (e.g. of a therapeutic response) in the subject.

One aspect of the invention is a kit for detecting whether a subject is at risk for developing heart failure, comprising one or more agents for detecting the amount of a protein of the invention. In some embodiments, other markers for heart failure (e.g., as discussed elsewhere herein) can also be present in a kit. The kit may also include additional agents suitable for detecting, measuring and/or quantitating the amount of protein, including conventional analytes for creation of standard curves. Among other uses, kits of the invention can be used in experimental applications. A person of ordinary skill in the art will recognize components of kits suitable for carrying out a method of the present invention.

If mass spectrometry is to be used to measure protein levels, the following reagents can be included in the kit: known amounts of a labeled (e.g. stable isotope) peptide (synthetic or recombinant) standard for each peptide to be assessed, separately or combined into a single mixture containing all peptides; optionally, a different peptide standard for assessing reproducibility of the assay; and/or, optionally, dilutant and trypsin for preparation of the sample. Kits for mass spectrometry are conventional and well-known in the art. A person of ordinary skill in the art will recognize components of kits suitable for detecting a biomarker(s) using mass spectrometry.

If an antibody-based method is to be used to measure protein levels, the agents in the kit can encompass antibodies specific for the proteins. In some embodiments, the antibodies are labeled with a detectable marker, e.g., a chemiluminescent, enzymatic, fluorescent, or radioactive moiety. In some embodiments, the kit includes a labeled binding partner(s) to the antibodies, Antibody-based kits for protein detection are conventional and well-known in the art. A person of ordinary skill in the art will recognize components of kits suitable for detecting a biomarker(s) using antibodies.

In some embodiments, a kit of the invention may comprise instructions for performing the method. Optionally, the kit can include instructions for taking a sample from the mammalian subject (e.g., body fluid), and using the kit to identify a mammalian subject at risk of developing heart failure. In some embodiments, a kit of the invention contains suitable buffers, containers, or packaging materials. The reagents of the kit may be in containers in which the reagents are stable, e.g., in lyophilized form or stabilized liquids. The reagents may also be in single use form, e.g., for the performance of an assay for a single subject.

Embodiments of the present invention can be further defined by reference to the following non-limiting examples, which describe the methodology employed to identify and characterize two novel phosphorylation sites on desmin that are linked to the molecular mechanism of heart failure. It will be apparent to those skilled in the art that many modifications, both to materials and methods, may be practiced without departing from the scope of the present disclosure.

### EXAMPLES

We used state-of-the-heart proteomic technologies to analyze both a canine and a human model of heart failure, and found new posttranslational modifications of desmin. It is understood that the examples and embodiments described herein are for illustrative purposes only and that various modifications or changes in light thereof will be suggested to persons skilled in the art and are to be included within the spirit and purview of this application.

### Example I

### Identification of Novel Cardiac Biomarkers for Heart Failure

### Canine Model of Heart Failure

The canine model of failure is well characterized, and was recently used to monitor the effects of bi-ventricular pacing, one of the few clinically effective therapies for HF. (Bax et al., J Am Coll Cardiol 46:2153-2167 (2005); and Bax et al., J Am Coll Cardiol 46:2168-2182 (2005)). Among the gross phenotypical changes that characterize the transition to failure in the mechanically challenged hearts of the DHF dogs, the disarrangement of desmin cytoskeleton is one of the most remarkable.

In our study, adult mongrel dogs (n=6) underwent either DHF or CRT protocols. Animals underwent left bundle-branch radiofrequency ablation to induce heart failure. See Chakir et al., Circ 117:1369-1377 (2008). Three animals were paced from the right atrium for six weeks at ˜200 bpm (DHF); whereas the remaining three dogs were subjected to three weeks of atrial pacing (dyssynchrony) followed by three weeks of bi-ventricular tachypacing at the same rate (CRT) as described in Bax et al., J Am Coll Cardiol 46:2153-2167 (2005). Left bundle branch block (LBBB) was confirmed by intra-cardiac electrograms, with surface QRS widening from 50±7 to 104±7 ms (p<0.001). Bi-ventricular pacing was achieved by simultaneous lateral epicardial and right ventricular antero-apical free wall stimulation. In addition, 3 adult mongrel dogs underwent sham operated control experiments.

At terminal study, the hearts were extracted under cold cardioplegia, dissected into endocardial and mid/epicardial segments from the septum (i.e., LV and RV septum) and LV lateral wall, and frozen in liquid nitrogen. Tissue samples obtained from the upper third of the LV lateral wall were used in the present study.

### Human Tissues

Human Left Ventricle (HLV) needle biopsies were obtained either from class III NYHA patients at the time of corrective surgery (valve replacement) or from healthy donors who died of causes other than heart failure.

### Sample Preparation

Tissue samples were snap frozen in liquid nitrogen at the time of dissection and stored at −80° C. Canine tissue samples were processed according to the IN-Sequence method developed by our laboratory and optimized for proteomics analysis as reported in Kane et al., Methods Mol Biol 357:87-90 (2007). Tissue specimens were directly homogenized in Hepes buffered medium (25 mM Hepes, pH 7.4, 1% w/vSDS, 0.1 mg/ml DNAse I, Protease inhibitor cocktail Complete, Roche). The same buffer was used to re-suspend the canine myofilament-enriched fractions. Protein concentration was determined by the BCA protein assay (Pierce), and 100-500 μg protein aliquots were prepared, snap frozen in liquid nitrogen and stored at −80° C. until further processing.

### Confocal Imaging

Because one of the earliest features of heart failure is myofilament disarrangement, tissue samples from the canine model of heart failure and bi-ventricular pacing were prepared for fluorescent microscopy, probed with anti-desmin antibody, phalloidin (actin) and DAPI (nuclei) and submitted to confocal imaging. Specifically, tissue samples were embedded in OCT right after dissection and stored at −80° C. Tissues were sliced by means of a cryostat set at 10 μm thickness and the sample sections transferred onto Superfros™ slides (Fisher) and probed with anti-desmin antibody (green), phalloidin (actin) (red), and DAPI (nuclei) (blue). Antibodies were diluted in 5% (w/v) milk in Tris-buffered saline (TBS) solution (1:1000 or 1:2500). Images were taken by means of a confocal microscope (Zeiss LSM 510 Meta), A 1000× magnification was achieved through oil immersion. Images were edited using ImageJ.

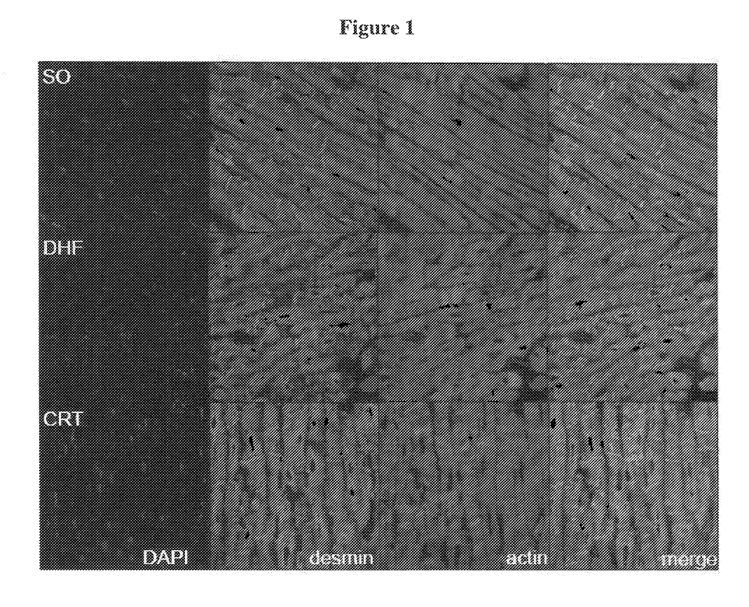

FIG. 1 shows representative images from these experiments. The results indicate that desmin cytoskeleton is disrupted in the failing hearts, as shown by the loss in organization (striation) in DHF samples. In particular, desmin seems to redistribute away from z-band and intercalated discs with DHF, in favor of a higher perinuclear distribution and lateralization. The trend is reverted when the animals are submitted to bi-ventricular pacing (Cardiac Resynchronization Therapy or CRT), a procedure commonly used in clinics to treat heart failure patients.

The Levels of Desmin PTM-Forms are Altered with Heart Failure

Based on our previous findings, desmin is posttranslationally modified in an in vitro model of cardiac hypertrophy. (Agnetti et al., Biochim Byophys Acta 1784:1068-76 (2008). To confirm that these observations are relevant in vivo, tissue specimens from failing (DHF) and sham operated (SO) canine hearts were subjected to IN-sequence fractionation to obtain a myofilament-enriched fraction containing desmin cytoskeleton. Myofilament-enriched fractions from DHF and SO were then analyzed using a classical Difference In-Gel Electrophoresis (DIGE) approach.

Sample protein profiles were compared by DICE using the myofilament enriched fraction (canine hearts) or the total protein homogenate (HLV). (Unlu et al., Electrophoresis 18:2071-77 (1997)). Protein extracts were labeled with different colored fluorescent dyes (CyDyes, GE healthcare), and different samples, including an internal standard (pool), were co-separated in the same two-dimensional electrophoresis (2DE) gel. This allows perfect superimposition of 2DE maps (particularly important for phosphorylation studies) and dramatically decreases technical variability. Raggiaschi et al., Proteomics 6:748-56 (2006); and Agnetti et al., Pharmacol Res 55:511-22 (2007). Cy3 or Cy5 dyes were used for individual samples and the dyes swapped for every condition to prevent bias due to dye affinity. For each gel set, a Cy2-labelled pool of all samples used in the assay was created (internal standard) by mixing equal amounts of protein from all the samples prior to labeling. Image analysis was contracted to Ludesi (Lund, Sweden), which further insured un-biased spot detection and matching.

Specifically, DICE analysis was performed using the protocol described in Kane et al., Proteomics 6:5683-87 (2006). The second dimension (SDS-PAGE) was run using 10% bis-tris gels with 2(n-morpholino) ethansulfonic acid (MES) running buffer. Graham et al., Proteomics 5:2309-14 (2005). Gel slabs were subsequently silver stained according to Shevchenko et al., Anal Chem 68:850-58 (1996). Sample pellets were diluted in isoelectric focusing (IEF) re-hydration buffer (8 mol/L urea, 2.5 mol/L thiourea, 4% w/v 3-[3-cholamidopropyl]-1-propane-sulfonate [CHAPS], 0.5% ampholytes, 50 mmol/L DTT, 1% HED, and 0.01% w/v bromophenol blue). IEF was carried out using a Protean® IEF cell (Bio-Rad). Immobilized pH gradient (IPG) Strips (18 cm pH 4-7 linear gradients) were actively rehydrated with the sample (150 μg of protein in 350 μL IEF buffer) at 50 V for 12 hrs, followed by a rapid voltage ramping consisting of 1 hr each at 300, 600, and 1000 V, followed by 10000 V for 45 kVh at 20° C. Proteins were separated in the second dimension by 10% Bis-Tris SDS-PAGE, using a MES running buffer (45 mmol/L, [2(N-morpholino) ethane sulfonic acid] or MES, 50 mmol/L Tris base, 0.1% SDS, 0.8 mmol/L EDTA, pH 7.3) as described previously 5. IPG strips were reduced and alkylated for 20 min each, respectively using 1% (w/v) DTT and 4% (w/v) iodoacetamide in equilibration buffer (50 mmol/L Tris-HCl, pH 8.8, 6 mol/L urea, 30% v/v glycerol, 9% w/v SDS). IEF strips were rinsed briefly with MES running buffer, the excess of liquid was gently removed with a paper tissue, and the strips were loaded onto the 10% Bis-Tris SDSPAGE gels. Strips were sealed using agarose sealing solution (50 mmol/L MES, 0.5% Agarose NA, 0.1% w/v SDS, bromophenol blue), Gels were run overnight on a Protean® H XL system (Bio-Rad) at 90 V. Gels were silver stained according to the protocol of Shevchenko et al. 6. Differential display analysis was contracted to Ludesi (Uppsala, Sweden).

A few proteins from the gel were also extracted and analyzed by mass spectrometry. Protein spots were excised from fresh gels, and destained according to a modified protocol of Gharandaghi et al., Electrophoresis 20:601-605 (1999). Proteins were digested in 25 mmol/L ammonium bicarbonate, pH 8.0 completed with 10 μg/mL sequencing grade modified porcine trypsin (Promega), for 16-24 h at 37° C. Peptides were extracted twice with 50 μL of acetonitrile (ACN) and 25 mmol/L ammonium bicarbonate 1:1 v/v for 60 min and then dried under vacuum. Tryptic peptides were reconstituted in 3 μL of 50% ACN/0.1% TFA and analyzed by electrospray ionization (ESI) MS/MS Deca XP Plus mass spectrometer (ThermoFinnigan, San Jose, Calif.), as described in Stastna at al., Curr Biol. 3:327-32 (1993).

Data-dependent acquisition was used to obtain both a survey spectrum along with several MS/MS spectra for multiply charged precursor ions present in each sample. MS/MS spectra were processed by baseline subtraction, and de-convoluted using Mascot wizard. Database searching was performed using Mascot wizard (www.matrixscience.com) using the “othermammalian” sub-database of NCBInr protein databases. PASTA sequences were blasted against Swissprot protein database through the proteomics tool Expasy Blast (http://www.expasy.ch/tools/blast/) to further reduce protein redundancy. The number of unique peptides assigned by Mascot search and retrieval system is also listed for each protein. The Mowse score provided by the software was manually recalculated (Corrected Mowse) summing unique peptides as defined in Wilkins et al., Proteomics 6:4-8 (2006), Observed and theoretical isoelectric point (pi) and molecular weight (MW) values for identified proteins are given, and these parameters were used to assign protein identities when ambiguous IDs were retrieved by Mascot.

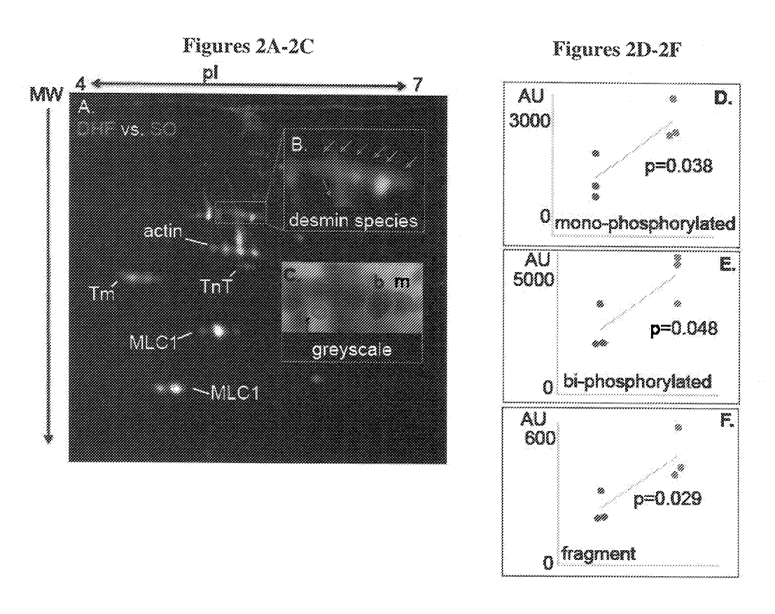

FIG. 2A is a representative DIGE gel containing SO (green), DHF (red) and internal standard (blue) samples. A few myofilament proteins were identified by MS/MS as well as several PTM-forms of desmin (indicated by arrows in FIG. 2B). The image analysis performed by Ludesi indicates that three desmin spots, compatible with a mono-phosphorylated, a bi-phosphorylated, and a fragment of desmin (FIG. 2C), were increased 2-fold in DHF hearts vs. sham operated animals (p<0.05, FIGS. 2D-2F).

### Altered Desmin Forms are Phosphorylated and Cleaved

To confirm the occurrence of desmin phosphorylation in the samples, the samples were subjected to alkaline phosphatase treatment as described in Agnetti et al., Circ Cardiovasc Genet. 3:78-87 (2010). Alkaline phosphatase (AP) removes negatively charged phosphate groups and induces a shift towards the basic side of a DIGE gel (to the right, by convention). In order detect the precise shift in pI, the AP treatment was coupled with DIGE analysis by substituting the internal standard with a pool of the samples treated with AP. Specifically, samples were re-suspended in 1% (w/v) SDS completed with protease inhibitor cocktail Complete™. The internal standard sample was then treated with alkaline phosphatase (CIP, New England Biolabs) overnight at 37° C. On the following day, the samples were solubilized in CHAPS buffer and labelled with CyDyes for 20 minutes at room temperature. The labeling reaction was stopped by adding 100 mM Lysine to the samples. Samples were flash frozen or diluted in IEF buffer for two-dimensional electrophoresis. DHF and CRT pools were alternatively labelled with either Cy3 and Cy5 (dye swapping) to prevent artifact variations due to dye bias.

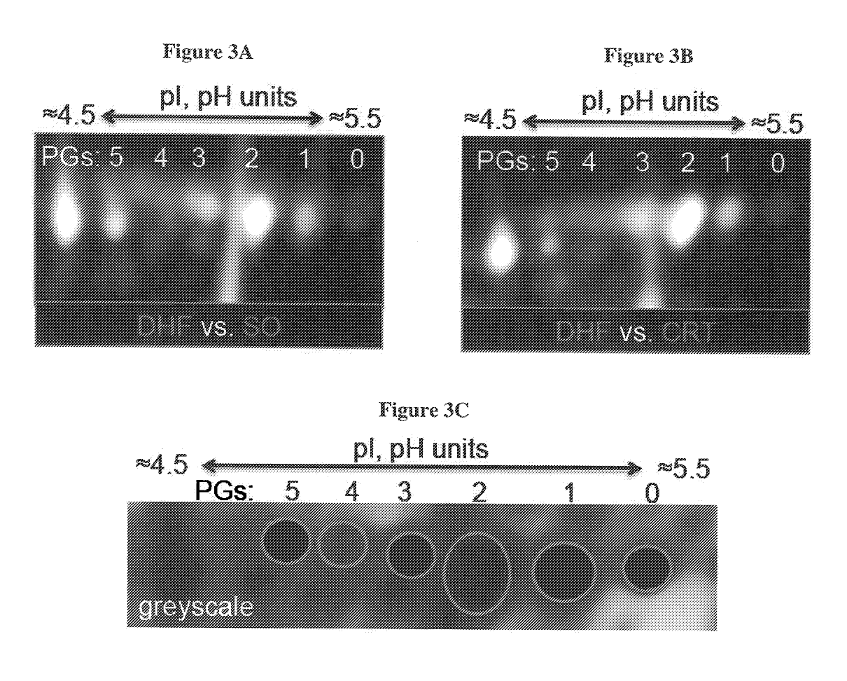

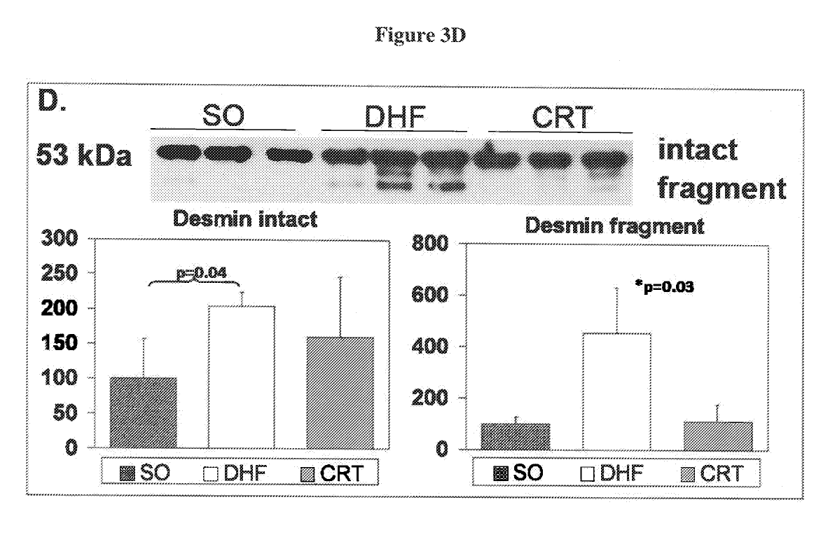

FIG. 3A shows a representative gel containing SO, DHF, and AP treated internal standard samples. Under these conditions, the increase in the color component assigned to the de-phosphorylated pool (blue in this case) on the basic (right) side of the gel as compared to SO (green) confirms the presence of desmin phosphorylation. The increase in the blue and red color components on the right side of the desmin isoelectric train confirms that the less phosphorylated forms of desmin (blue) are more abundant in DHF (red). Intriguingly, this trend is reverted when DHF are compared to CRT animals, suggesting that the presence of these low phosphorylated forms of desmin are detrimental to a subject's heart and are biomarkers of heart failure (FIG. 3B).

The number of phosphate groups (PGs) in FIG. 3 was assigned assuming that the most basic form of desmin after de-phosphorylation is the un-phosphorylated form. FIG. 3C shows a magnified gel image in grayscale were desmin phospho-forms are highlighted and PG numbers are reported.

Samples were also analyzed by Western blot. Proteins were transferred to PVDF in transfer buffer at 100 V for 1 hour in ice. Membranes were stained with Direct Blue 71 (Sigma), and images recorded for subsequent luminescent signal normalization. Membranes were then blocked overnight using 5% milk in Tris-buffered saline (TBS: 100 mmol/L Tris-Cl, 0.9% (w/v) NaCl) completed with 0.1% Tween 20 (TBS-T); and incubated with 0.2 μg/mL anti-desmin antibody mouse IgG monoclonal in TBS-T under gentle agitation for 1 hr, and then incubated with 0.03 μg/mL alkaline phosphatase conjugated AffiniPure Goat Anti-Mouse (Jackson ImmunoResearch) in TBS-T under gentle agitation for 1 hr. Chemiluminescent signal was produced using Immun-Star AP substrate pack (BioRad Laboratories) and luminescence was detected with scientific imaging film (Kodak).

FIG. 3D is a representative western blot containing DHF, SO, and CRT samples probed with a desmin specific antibody. Interestingly, a desmin fragment was increased in DHF samples as compared to both CRT and SO samples (4-fold, p<0.03). Our findings suggest that desmin cleavage is maladaptive and is another marker of heart failure

### Desmin Phosphorylation Status is Modified in Class III NYHA Patients

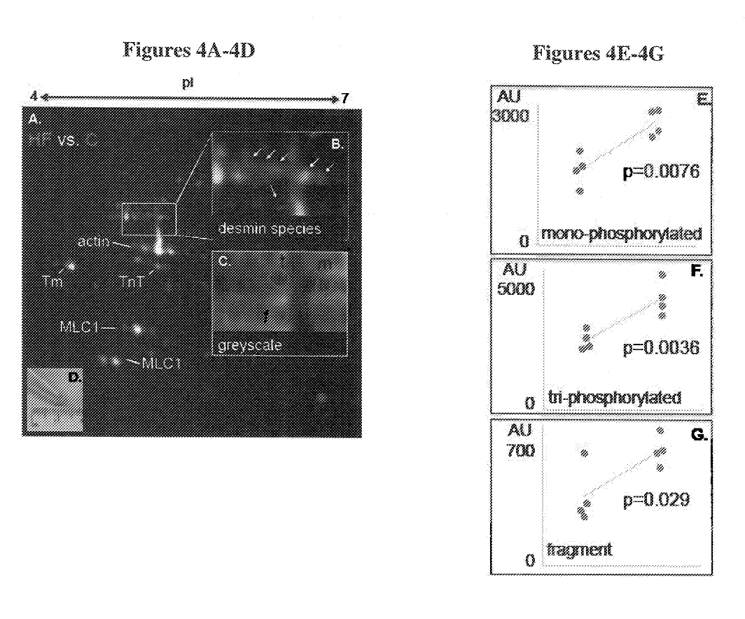

We also subjected human heart biopsies from the LV of heart failure patients and normal donors to a classical DICE comparison. Humans HLV needle biopsies (˜3 mg) were homogenized and the total protein extracts were subjected to DIGE analysis. FIGS. 4A and 4B show a representative gel containing samples from heart failure patients and healthy subjects. A relative grayscale image is provided in FIG. 4C. The differential display analysis performed by Ludesi indicated that at least three forms of desmin are increased in heart failure patients (FIGS. 4D-4E). According to their electrophoretic mobility, these spots are compatible with a mono-phosphorylated, a tri-phosphorylated, and a fragment of desmin (2-fold, p<0.03). Other desmin forms were also statistically increased but to a smaller extent.

These findings confirm the clinical significance of decreased levels of desmin phosphorylation in heart failure.

### Desmin is Phosphorylated at Ser-27 and Ser-31

We further assessed desmin phosphorylation using phospho-peptide enrichment techniques (IMAC) and tandem MS. Agnetti et al., Pharmacol Res 55:511-522 (2007).

Gel slabs were post blue-silver stained according to Candiano et al., Electrophoresis 25:1327-33 (2004). Protein spots were collected and hi-gel digested for subsequent MS analysis. A Maldi-T of/T of mass spectrometer (4800, Applied Biosystem Inc.) was used for identification whereas an LC-Q ion-trap (Thermo) was employed for the characterization of desmin phosphorylated sites upon phosphopeptides enrichment.

Phosphopeptides were enriched with an Immobilized Metal Affinity Chromatography (IMAC) column essentially as described by Ficarro et al., Nat Biotechnol 20:301-5 (2002); and Arrell et al., Circ Res 99:706-14 (2006). The reported phosphopeptide sequence was confirmed by manual inspection of the MS/MS spectra. The human phosphorylation sites were confirmed by means of an Orbitrap (Thermo) tandem MS.

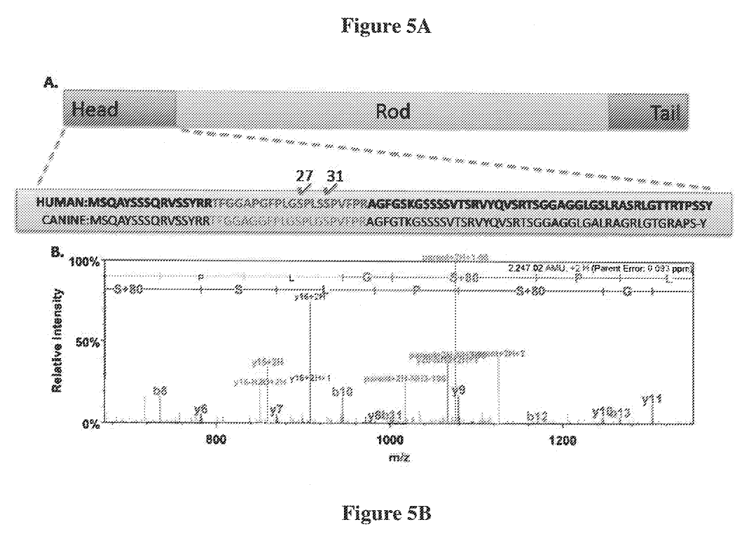

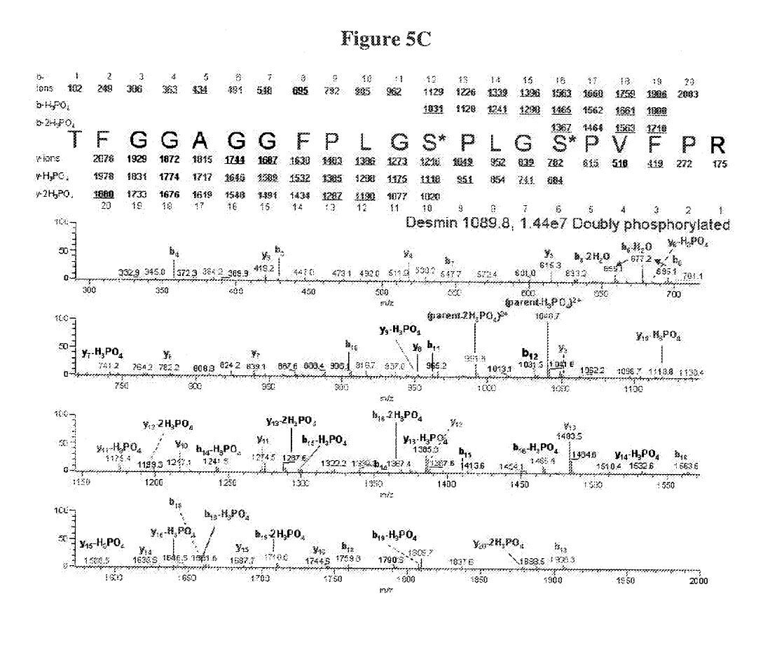

FIG. 5A shows the sequence of human and canine desmin. FIG. 5B is a representative MS/MS spectrum for human desmin, and FIG. 5C is a representative MS/MS spectrum for canine desmin, Two novel phosphorylation sites were found in the N-terminal domain of human and canine desmin: Ser-27 and Ser-31, which are each in the N-terminal head domain of desmin, a portion of the protein known to be critical for its in vitro susceptibility to PTMs and for its role in mature IFs assembly.

In canine samples, the monophosphorylated peptide TFGGAGGFPLGS*PLGSPVFPR was detected only in DHF samples whereas the bi-phosphorylated peptide (m/z=2179.6) was found in both sham and DHF dogs. This observation is the above DICE analysis showing the increase in the levels of desmin forms with low phosphorylation status (mono- and bi-phosphorylated) during heart failure.

### Multiple Reaction Monitoring of Human Phospho-Desmin.

We optimized a multiple reaction monitoring (MRM) protocol to measure the singly phosphorylated peptide in clinical samples. The strength of this technique relies mainly on its sensitivity and specificity; it is also unbiased, unlike alternative techniques such as immunostaining. Indeed, modified proteins may display a different immunoreactivity depending on their PTM status.

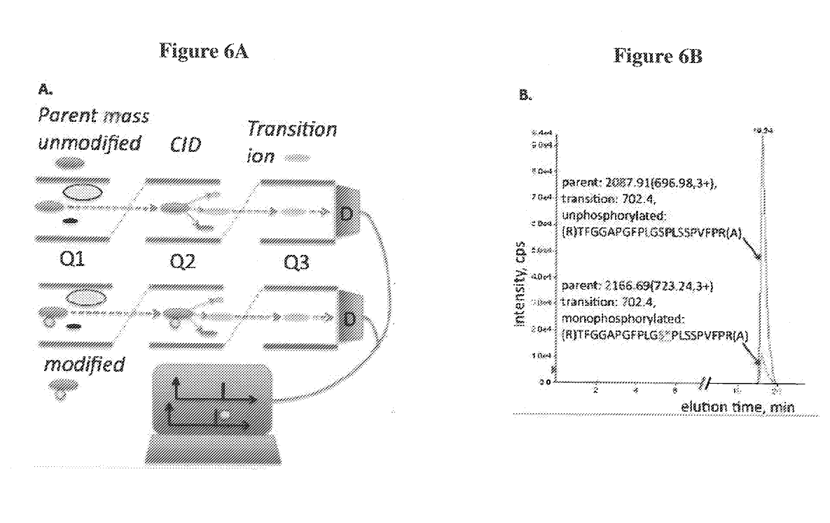

A schematic of the MRM protocol is depicted in FIG. 6A. MRM analysis requires protein digestion into peptides, which can be performed downstream of a 1DE separation, using purified protein bands. Peptides (modified and unmodified) have a specific mass, and these values can be used to select a specific peptide ion (parent) in the first analyzer (or quadrupole, Q1) of the MS (triple quadrupole or Q³), The selected peptide species can be fragmented in the second selector (Q2), and its fragments (or transition ions) can be monitored in the third analyzer (Q3). The intensity of the peaks can be normalized using an internal standard (purified, custom peptide, alternatively labeled with heavy isotopes) and used for quantitation.

FIG. 6B is a representative MRM chromatogram showing the relative abundance of un- and mono-phosphorylated desmin (Ser-27) in human samples.

### Desmin-Positive Oligomers are Increased in Heart Failure

Desmin IFs tensile strength was recently measured by AFM and found to be in the range of 10² MPa, (Kreplak et al., J Mol Biol. 385:1043-51 (2009). Desmin filaments are capable of resisting lateral forces as high as 40 MJ/m³ at 240% extension, whereas actin filaments can only face 0.5 MJ/m³ before they break. These observations support the view that IFs cytoskeleton is likely responsible for maintaining cell integrity and mechanic unity under stressed conditions, such as those observed in the dyssynchronous heart or other forms of heart failure, (Kreplak et al., Biophys J 94:2790-2799 (2008). However, when IFs filaments are stretched beyond their physical capacity, they irreversibly lose their conformation and generate the same beta-sheet structures that are observed in amyloid-like species. Kreplak et al., J Mol Biol 354:569-577 (2009). For these reasons, we investigated the effect of desmin modification on its assembly by BN-PAGE.

Desmin oligomers were separated by blue-native (BN) PAGE in the presence of 2% SDS. Stegemann et al., Proteomics 5:2002-9 (2005). Myofilament-enriched fractions from IN-Sequence were diluted in EN-sample buffer (25 mM BisTris, 0.015 N HCl, 10% glycerol, 25 mM NaCl, 0.001% Ponceau S) completed with 2% SDS and 0.5% Coomassie Brilliant Blue (CBB) 0250, and then incubated for 30 min at RT. After “solubilization” of the oligomers, samples were centrifuged at 18000 ref and separated on precast Native-PAGE gels (Invitrogen) for 1 hour 30 min at 150 V according to manufacturer instructions, CBB 0250-stained gel images were recorded for downstream protein load normalization, and gels were either fixed overnight for MS analysis or blotted onto PVDF membranes for western blotting as described herein.

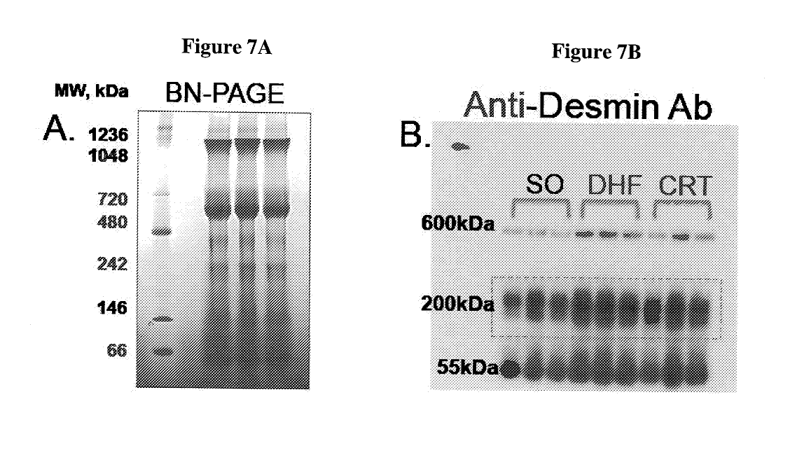

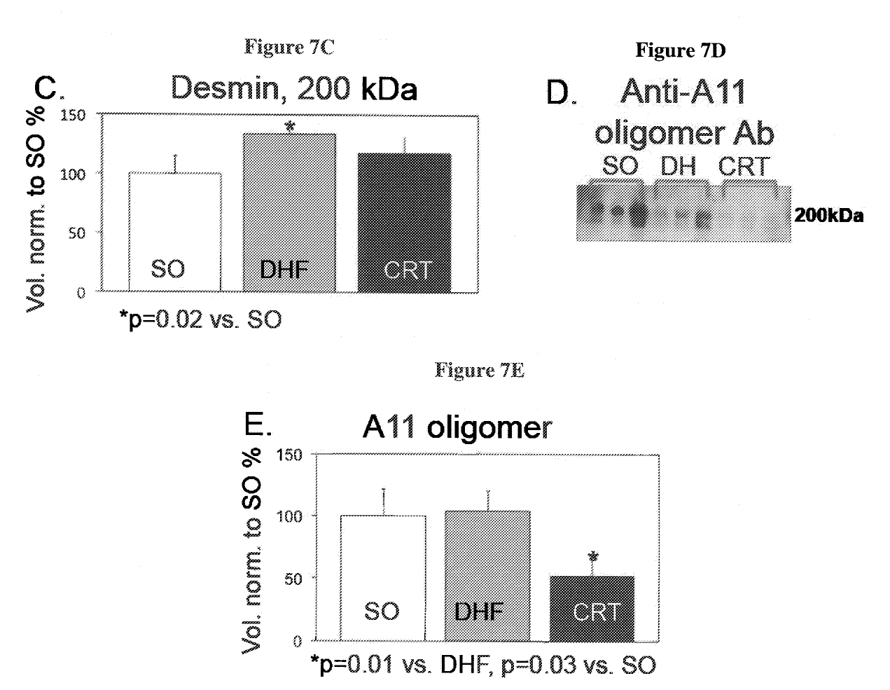

FIG. 7A is a representative image of such a BN-PAGE gel. The presence of desmin in these oligomers was assessed by western blot analysis using an anti-desmin antibody (DE-U-10, Sigma, mouse, monoclonal) (FIG. 7B). This anti-desmin antibody detected three major bands at approximately 50, 200 and 600 kDa. These are compatible with the monomer and two oligomeric forms of desmin. Densitometric analysis revealed that all three desmin forms were increased in DHF animals compared to sham (˜50 kDa: 27.3±4.9SD; ˜200 kDa: 33.4±4.2SD; 400 kDa: 52.4±10.4SD, all p<0.03; FIGS. 7B and D).

After stripping and re-probing with a rabbit anti-A11 oligomer antibody (Invitrogen), at least one band was detected at ˜200 kDa, perfectly superimposed to the desmin signal (FIG. 7C). As this antibody is able to recognize a toxic domain common to different amyloid oligomers (Kayed et al., Science 300:486-89 (2003); and Glabe al., J Biol Chem 283:29639-643 (2008)), these results suggest that at least part of the ˜200 kDa desmin oligomer contains this toxic amyloid domain, Intriguingly. CRT was able to lower the levels of these species, suggesting that the beneficial effects of this therapy could be mediated by the reduced formation of amyloid species in viva (FIG. 7E).

As such, we have discovered a novel mechanism of heart failure based on the formation of toxic, amyloid species.

### Example II

### Identification/Generation of Desmin Antibodies

The discovery of the desmin phosphorylation as a molecular mechanism of heart failure has important implications in treating a subject at risk for developing heart failure. Utilizing our finding that Ser-27 and Ser-31 are critical phosphorylation residues in desmin, one can develop reagents such as antibodies that target these residues. Antibodies to these residues in various states of phosphorylation (e.g., un-, mono-, bi-, and tri-phosphorylation) will be generated and used to practice the various embodiments of the present invention described herein.

Antibodies can be made using conventional techniques that are well-known in the art. See supra. For example, one can employ use of hybidoma techniques. In this approach one immunizes animals with a particular form of desmin (e.g., the TFGGAGGFPLGSPLGSPVFPR desmin peptide phosphorylated at Ser-27 and/or Ser-31). Hybridomas can then be developed from these animals using standard techniques. One can then screen these hybridomas by ELISA or other techniques to identify those hybridomas that produce antibodies that recognize the particular form of desmin.

All publications, patents, patent applications, internet sites, and accession numbers/database sequences (including both polynucleotide and polypeptide sequences) cited herein are hereby incorporated by reference in their entirety for all purposes to the same extent as if each individual publication, patent, patent application, internet site, or accession number/database sequence were specifically and individually indicated to be so incorporated by reference

1. An antibody that specifically recognizes phosphorylated serine 27 and/or phosphorylated serine 31 in desmin.

2. The antibody of claim 1, wherein the antibody is a monoclonal antibody.

3. The antibody of claim 1, wherein the antibody is a polyclonal antibody.

4. The antibody of claim 1, wherein the antibody is labeled.

5. The antibody of claim 4, wherein the label is a fluorescent label, a moiety that binds another reporter ion, a heavy ion, a gold particle, or a quantum dot.

6. A kit for identifying a subject at risk for developing heart failure, comprising at least one agent that detects the phosphorylation state of a desmin protein at serine 27 and/or phosphorylated serine 31.

7. The kit of claim 6, wherein the agent is an antibody that recognizes the phosphorylation state of serine 27 and/or phosphorylated serine 31.

8. The kit of claim 6, wherein the agent is an antibody that recognizes un-, mono, di-, and/or tri-phosphorylated serine 27.

9. The kit of claim 6, wherein the agent is an antibody that recognizes un-, mono, di-, and/or tri˜phosphorylated serine 31.

10. The kit of claim 6, wherein the agent is in a container.

11. The kit of claim 6, further comprising instructions for taking a biological sample from the subject.

12. A method for identifying a subject at risk for developing heart failure, comprising:
(a) obtaining a biological sample from the subject;
(b) measuring the level of at least one biomarker in the biological sample, wherein the biomarker comprises a desmin protein; and
(c) comparing the level measured in the biological sample to a control level in a normal subject population;
wherein a decrease in phosphorylation of serine 27 or serine 31 in the desmin protein, compared to the control level, is indicative that the subject is at risk for developing heart failure.

13. A method for treating a subject at risk for developing heart failure, comprising:
(a) obtaining a biological sample from the subject;
(b) measuring the level of at least one biomarker in the biological sample, wherein the biomarker comprises a desmin protein;
(c) comparing the level of phosphorylated serine 27 or serine 31 in the desmin protein to a control level in a normal subject population; and
(d) treating a subject having decreased levels of phosphorylation to reduce risk of heart failure.

14. The method of claim 12, wherein the biological sample is blood, plasma, or serum.

15. The method of claim 12, wherein the biological sample is cardiac tissue, tissue homogenate, or tissue slice.

16. The method of claim 12, wherein the biomarker(s) is detected using mass spectrometry.

17. The method of claim 16, wherein the mass spectrometry is multiple reaction monitoring.

18. The method of claim 12, wherein the biomarker(s) is detected using an immunoassay.

19. The method of claim 12, wherein treating a subject having decreased levels of phosphorylation comprises administering aggressive therapy to the subject.

20. The method of claim 19, wherein the aggressive therapy is cardiac resynehronization therapy.

21. A method for treating a subject at risk for developing heart failure, comprising:
(a) obtaining a biological sample from the subject;
(b) measuring the level of at least one biomarker in the biological sample, wherein the biomarker comprises a desmin protein;
(c) comparing the level of phosphorylated serine 27 or serine 31 in the desmin protein to a control level in a normal subject population; and
(d) treating a subject having normal levels of phosphorylation with non-aggressive therapy.

22. The method of claim 12, further comprising detecting the level of a second biomarker for heart failure,

23. The method of claim 22, wherein the second marker is cardiac specific isoforms of troponin I (TnI) or troponin T (TnT), CK-MB, myoglobin, or brain natriuretic peptide (BNP).

24. The method of claim 23, wherein the second marker is brain natriuretic peptide (BNP).

25. The method of claim 12, which is a method for following the progression of myocardial infarction or ischemia in the subject.

26. The method of claim 12, wherein the detection is carried out both before or at approximately the same time as, and after, the administration of a treatment, and which is a method for determining the effectiveness of the treatment.

27. The method of claim 12, wherein the subject is a mammal.

28. The method of claim 27, wherein the subject is a human, dog, or horse.

29. A method of detecting desmin phosphorylation at serine 27 and/or serine 31 comprising:
(a) obtaining a test sample; and
(b) contacting the test sample with an antibody that specifically recognizes phosphorylated serine 27 and/or serine 31.

30. The method of claim 29, wherein the method is an immunoassay.

31. The method of claim 30, wherein the test sample is a histological preparation of a biopsy sample from cardiac tissue.

32. The method of claim 31, further comprising the step of visualizing the test sample by immunohistochemicai staining after step (b).

33. The method of claim 29, wherein the method is mass spectrometry.
